(19) United States
(12) Patent Application Publication
(10) Pub. No.: US 2005/0238181 A1
(43) Pub. Date: Oct. 27, 2005

(54) Hearing protector

(76) Inventors: Sigvard Nilsson, Varnamo (SE); Henrik Nordin, Forsheda (SE)

Correspondence Address: CANTOR COLBURN, LLP, 55 GRIFFIN ROAD SOUTH, BLOOMFIELD, CT, 06002, US

(21) Appl. No.: 10/999,367

(22) Filed: Nov. 29, 2004

Related U.S. Application Data
(60) Provisional application No. 60/608,616, filed on Sep. 10, 2004.

(30) Foreign Application Priority Data
Nov. 27, 2003 (SE) 0303189-5

(51) Int. Cl. A61F 11/06; G10K 11/16; H03B 29/00

(52) U.S. Cl. 381/72; 381/71.6

(57) ABSTRACT

A hearing protector device is provided including a sound attenuating body and an electronic unit having a microphone, an amplifier, and a loudspeaker, where the electronic unit is selectively activated and, when activated, receives sound from an ambient environment, amplifies a frequency range of the received sound corresponding to a frequency range of human speech, and varies said amplification such that a total level of sound passing through the body and through the electronic unit does not exceed a maximum predetermined sound level.

### CROSS REFERENCE TO RELATED APPLICATIONS

This application is related to and claims the benefit of U.S. Provisional Patent Application No. 60/608,616 filed on Sep. 10, 2004, which is herein incorporated by reference in its entirety. Also, this application claims the benefit of Swedish Patent Application No. 0303189-5 filed on Nov. 27, 2003, which said Swedish application is also herein incorporated by reference in its entirety.

### TECHNICAL FIELD OF THE INVENTION

The present invention relates to hearing protection and, more particularly, to a hearing protector device which provides protection from ambient noise but still allows a user to hear sounds relating to communication.

### BACKGROUND OF THE INVENTION

Hearing protectors are often employed in noisy environments, such as factories, airports etc. Such hearing protectors consist of two muffs or hoods manufactured from hard material, for example plastic, which sealingly surround the ears of the wearer and which are connected to one another by the intermediary of a headband. Interiorly in the muffs or hoods, there is provided as a rule a porous noise absorbent agent. The employment of hearing protectors has increased at the same rate as awareness has grown of the risks of loss of hearing caused by noise, and developments are constantly being made of the hearing protectors which occur on the market in order to make them more user-friendly, so that the desired level of use is encouraged. The majority of hearing protectors utilize so-called passive damping, i.e. a damping that takes place with the aid of the material included in the muffs or hoods and noise absorbent agents disposed in them.

A remaining problem in the employment of hearing protectors is that there is often a need to listen to requisite sound, such as instructions, or other conversations from a person in the vicinity, at the same time as the intention is to impede as much background noise as possible.

Hearing protectors occur on the market which, in response to the level, damp noise above a certain sound level. In this level-dependent damping, the hearing protector allows the passage of all sound up to a pre-determined sound level, with the aid of a microphone, an amplifier and a loudspeaker disposed inside the hearing muff or hood. When this level is exceeded, the electronics are shut off and all sound is damped by passive damping.

There also occur different types of communication systems in connection with hearing protection. For example, all people in factory premises may be provided with hearing protection which includes a radio receiver. The radio transmissions that can be received are often standard radio channels, but may also include or consist of a local transmission at the company in question. By such means, centrally transmitted instructions, alerts and other information are received as a one-way communication, but the system does not satisfy the need for communication between the individual recipients.

A problem common to existing hearing protectors is that they are not used in those cases where they are experienced as being inconvenient to use. Instead, there is a tendency that the user quite simply removes the hearing protector when s/he wishes to talk to someone else, for example to receive instructions or the like. Thereafter, there is a major risk that the wearer forgets to replace the hearing protector, with a consequential increase in the risk of loss of hearing through noise.

### BRIEF SUMMARY OF THE INVENTION

The above discussed and other problems and deficiencies of the prior art are overcome or alleviated by the invention which provides a novel and nonobvious hearing protector device.

The invention provides a hearing protector which affords the possibility of selective communication, where the hearing protector is so simple to use that the risk of loss of hearing through ambient external noise is minimized and where the audible sensitivity to speech is maximized when communication is desired.

A hearing protector device is provided including a sound attenuating body and an electronic unit having a microphone, an amplifier, and a loudspeaker, where the electronic unit is selectively activated and, when activated, receives sound from an ambient environment, amplifies a frequency range of the received sound corresponding to a frequency range of human speech, and varies said amplification such that a total level of sound passing through the body and through the electronic unit does not exceed a maximum predetermined sound level.

A hearing protector is also provided herein as including a hearing hood with passive noise damping, a microphone disposed exteriorly on the hearing protector, a loudspeaker disposed inside the hood; and an amplifier for amplifying sound signals caught by the microphone and passing the signals on to the loudspeaker, where the noise damping of the hearing hood is broad-band, the amplifier has variable amplification and frequency range which corresponds to the frequency range of human speech, and the amplifier has a maximum predetermined amplification where the sum total of the sound levels that are caused, on the one hand by ambient sound passing through the hood, and on the other hand by sound emitted by the loudspeaker, amounts to a maximum predetermined value.

The above-discussed and other features and advantages of the apparatus and method will be appreciated and understood by those skilled in the art from the following drawings and detailed description.

### BRIEF DESCRIPTION OF THE DRAWINGS

Referring now to the drawings wherein like elements are numbered alike in the several FIGURES:

FIG. 1 is a perspective view of a hearing protector according to the present invention;

FIG. 2 is a perspective view of a lid for a removable unit included in the hearing protector, a battery hatch not being shown in the Figure;

FIG. 3 is a perspective view of the battery hatch which is omitted from FIG. 2;

FIG. 4 is a perspective view of a hearing muff or hood included in the hearing protector, where the removable unit has been removed;

FIG. 5 is a perspective view of a unit intended for mounting in a hearing hood, in a second embodiment of the present invention;

FIG. 6 is a perspective view of the hearing protector shown from the inside, with the unit of FIG. 5 mounted in position;

FIG. 7 is a perspective view of a hearing protector in another embodiment of the present invention;

FIG. 8 is a perspective view of a hearing protector in another embodiment of the present invention;

FIG. 9 is a perspective view of a hearing protector in another embodiment of the present invention;

FIG. 10 is a perspective view of a hearing protector in another embodiment of the present invention;

FIG. 11 is a perspective view of a hearing protector in another embodiment of the present invention; and

FIG. 12 is a perspective view of a hearing protector in another embodiment of the present invention.

### DETAILED DESCRIPTION OF THE INVENTION

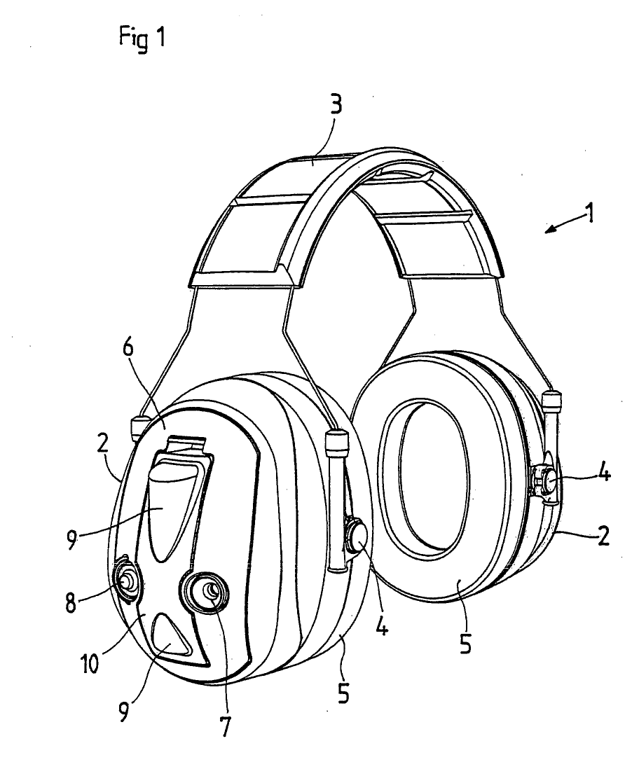

FIG. 1 shows a hearing protector 1 according to the present invention. The hearing protector 1 includes, in the usual manner, two hearing protector muffs or hoods 2 and a stirrup shaped headband 3 which unites the two hoods 2. On each side of each respective hood 2, there is provided a pivot 4 which connects the hoods 2 pivotally to the headband 3.

The hoods 2 have hard outer shells and a damping interior which may be provided in a manner that is previously known in the art. For example, the hoods 2 may be provided with double shells or some other type of passive noise damping. Further, the hoods 2 each display a sealing ring 5 for abutment against the head of the user around the user's ears. The sealing rings 5 may be designed in any optional manner which is previously known in the art.

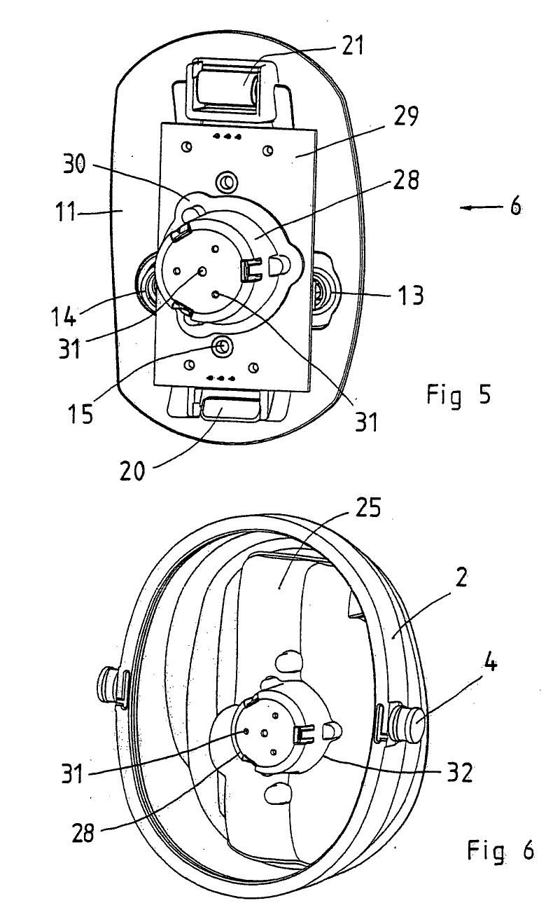

According to the present invention, at least the one hood 2 has a unit 6 which makes for communication to the person wearing the hearing protector. The unit comprises a microphone 7 disposed exteriorly on the hood 2, an electronics unit with an amplifier with variable amplification and possibly adjustable band width, as well as a loudspeaker 28 (FIGS. 5 and 6) disposed interiorly in the hood. The unit, which may be removable, but also integrated in the hood 2, further includes a battery hatch [0 which covers a recess 12 and which has a bulge 9 for accommodating a battery (not shown).

The microphone 7 is preferably a microphone possessing no particular directional effect, in other words a so-called broadcast microphone 7. This implies that the microphone 7 per se has the capability to take up sound regardless of the position of the source of the sound in relation to the microphone 7. The positioning of the microphone 7 should also be such that its function is not essentially affected by the direction in which the hearing protector 1 is turned for maximum flexibility in the use thereof. In this case, the microphone, as is clearly apparent from FIG. 1, is laterally directed in relation to the head of the wearer of the hearing protector.

In certain cases, a forwardly directed positioning of the microphone 7 may be preferred. In this embodiment, the microphone is directionally active and is turned to face towards a person standing in front of and speaking to the wearer of the hearing protector.

An activator button 8 is provided and is designed so that it is readily accessible by the user. The positioning of this button should also be such that it functions equally well regardless of in which direction the hearing protector is facing. This implies that a satisfactory function of one and the same hearing protector will be attained regardless of whether the user is right-handed or left-handed or whether the user, for one reason or another, wears the hearing protector back-to-front.

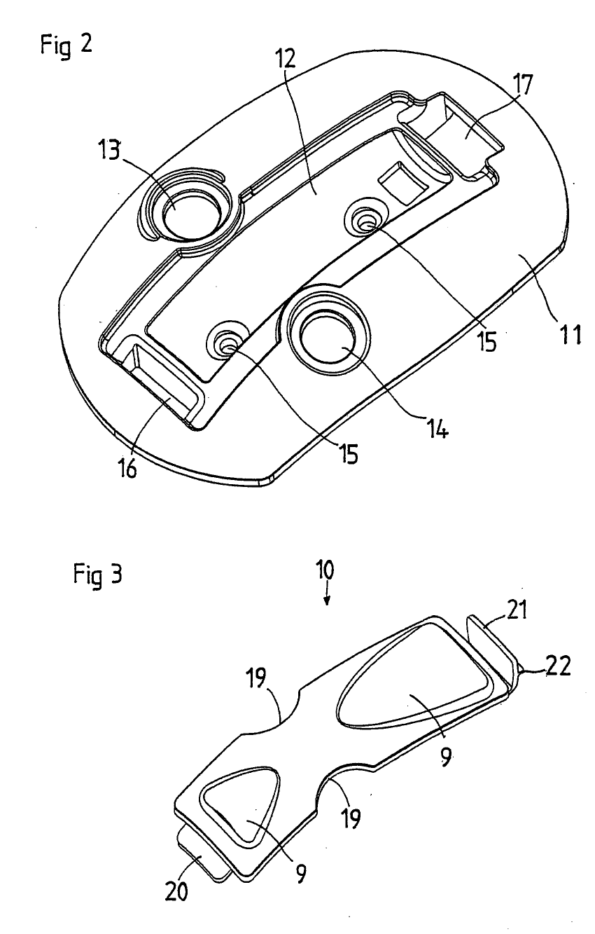

FIG. 2 shows a covering plate for the removable unit 6. The covering plate or lid has a recess 12 in the form of a depression for accommodating a battery. In the bottom of the recess 12, there are provided, in the preferred embodiment, two holes 15 for screws or similar fixing devices such as, for example, flanging. Above and below the recess 12, there are provided two substantially rectangular depressions 16 arid 17. These depressions are intended for fixing a hatch 10 which covers the battery and which displays the above-mentioned bulge 9.

Further, the covering plate 11 has a recess 13 for accommodating the operating device in the form of the activator button 8. A similar recess 14 is disposed on the opposing side of the battery recess 12 for accommodating the microphone 7 provided in the removable unit 6.

The removable unit 6 also includes a loudspeaker and electronic components for realizing both amplification and broad-band restriction of incoming sonic signals and a timer function for shutting off the electronics after a given predetermined time.

FIG. 3 shows the battery hatch 10 which is intended to cover the battery when this is housed in the recess 12. The battery hatch 10 has bulges 9 which correspond to the configuration of the battery in those regions which extend outside the major plane of the covering plate 11 and the battery hatch 11. The hatch further displays lower 20 and upper 21 fixing members for an openable fixing of the battery hatch 10. The lower fixing member 20 is a substantially planar projection which is intended to be received in a corresponding recess in the covering plate 11. the upper fixing member 21 displays a substantially U-shaped cross section, whose free shank may be actuated in a direction towards the battery hatch 10 for releasing the hatch 10 from the covering plate 11. On its side facing away from the battery hatch 10, the upper fixing member 21 has a longitudinal, catch-shaped member 22 which is disposed to pass the edge of the depression 17 when the upper fixing member is actuated inwardly in a direction towards the battery hatch 10. The catch-shaped member 22 thus snaps in behind the edge of the depression 17.

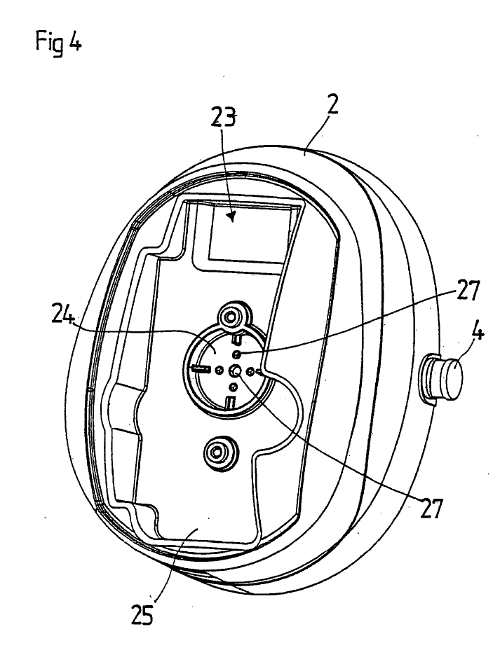

FIG. 4 shows one of the hearing hoods 2 seen from the outside and without the removable unit 6. Thus, the hearing hood 2 has a quite large recess 23 for receiving the removable unit 6. The recess 23 has a bottom wall 25 and is designed so as to afford room for the removable unit 6 with, for example, its recess 13 for the actuator member, the recess 14 for the microphone 7 as well as the depressions for the fixing members 20, 21 of the battery hatch 10. In the bottom 25 of the recess 23, there is further provided a perforated recess 24 for the loudspeaker facing towards the inside of the hearing hood 2. In principle, the bottom 25 functions as a partition between the recess 23 and the inside of the hearing hood 2.

The fact that the perforations 27 are placed in the bottom of the recess 23 implies that they are also placed close to the opening of the acoustic meatus of the wearer. This entails that the sound from the loudspeaker will be perceived as more dominant in relation to the ambient noise which penetrates through the hearing hood, for which reason the amplification by the amplifier may be made weaker without any deterioration of the audibility of the spoken word emitted by the loudspeaker. This implies less electric power consumption and consequentially a longer service life for the batteries.

The properties of the bottom 25 correspond to those of the outer defining wall of the hearing hood 2, i.e. the removable unit 6 is functionally disposed on the outside of the hearing hood 2, while, in terms of appearance, it is recessed in the hearing hood 2. Consequently, in order to keep the bottom tight against the outside and prevent noise coming from outside from being allowed through the perforation 27, the portion of the removable unit 6 around the loudspeaker sealingly abuts against the edge of the depression 24. The sealing abutment may be realized in any optional manner which is previously known in the art.

Since the bottom 25 is to be considered as a part of the defining wall of the hood 2, no further seals are required between the removable unit 6 and the edge of the recess 23. Nor is it necessary to provide any special sealing at the battery hatch 10.

The removable unit 6, which is not shown in FIG. 4, may be designed as a standard unit. This implies that the same removable unit 6 may be employed together with a large number of different hoods 2 possessing different passive damping properties, which reduces the need for storekeeping of parts and provides a possibility to adapt the hearing protector in response to different noisy environments.

The hearing protector 1 functions as follows:

On those occasions when the user wishes to listen to someone or something, the activator button 8 is depressed, which activates the microphone 7 and the associated amplifier which transmits sound to the inwardly directed loudspeaker. The sound taken up by the microphone 7, i.e. preferably speech, is amplified to a suitable sound level inside the hearing protector. Any possible background noise is amplified as little as possible. When the communication is completed and the reception of more sound signals from the microphone 7 is no long required, the electronics are deactivated by the release of the activator button 8. An alternative to manual deactivation is that the electronics in the hearing protector 1 automatically shut off the loudspeaker after a given time lapse.

The automatic shut off is realized in practice with the aid of a special delay circuit which, after a predetermined time lapse from activation once again shuts off the electronics of the hearing protector. In the preferred embodiment, the time delay is about 20 seconds. If the intention is to interrupt communication earlier, the electronics are manually shut off by once again depressing the activator button 8. A variation where the activator button 8 is held depressed continuously for a longer period of time and the automatic shut off is disabled while the button is held depressed is less satisfactory since the deactivation function would then be rendered inoperative, for example in that the button 8 is taped down.

The electronics in the unit are formed in such a manner that they display a variable amplification of the incoming signal via the microphone 7, where the level of amplification is varied so that the higher the sound level that is sensed by the electronics, the lower will be the level of amplification. The amplification is limited to a value where the level of the sound emitted by the loudspeaker together with the level of the ambient noise that penetrates through the protector hood amounts to such a maximum predetermined value as is considered tolerable in view of the risk of loss of hearing due to noise. If the penetrating ambient noise alone reaches this value, the electronics are kept disabled so that the loudspeaker is silent.

The hearing protector hood per se displays passive noise damping with large bandwidth. As a result, ambient noise is damped within a broad frequency range. Customized adaptation to certain noisy environments, with particularly good damping in certain frequency bands can naturally be put into effect. One example might be noisy environments with very low frequencies.

The protector hood 2 also displays frequency limitation for those frequencies that are amplified via the electronics. The interesting range for understanding the spoken word extends from 125 to 6000 Hz. The focal point lies between these extreme limits, for which reason the preferred system of microphone 7, loudspeaker 28 and amplifier preferably operates in the range of from about 500 Hz to 3500 Hz, i.e. within the frequency range of human speech. These limits are merely exemplified, for which reason the figure values may vary, depending on the steepness of the filters which are employed for the frequency limitation.

According to the present invention, it is possible to vary, within the pass band of the electronics, the amplification according to frequency so that a “equalizer effect” is achieved and as a result the understanding of spoken sound transmitted via the electronics will be optimized.

The hearing protector 1 possesses, as was mentioned above, passive damping. The passive damping is frequency-responsive and is affected by the appearance, design and other properties of the hood 2. In order for the present invention to be usable, the protector hood 2 should be of such a type that in itself damps noise effectively.

In the preferred embodiment, a given configuration of the unit 6 was shown. It is naturally possible to vary the appearance of this unit in innumerable different ways, as well as to design it as an integral part of the hood proper.

FIGS. 5 and 6 show an alternative embodiment of the unit which per se includes means for frequency-responsive amplification of the signal taken up by the microphone 7 disposed on the outside of the protector hood 2. It will be apparent from the Figure that the unit includes a covering plate 11 which, on its rear side (inside), carries a circuit card 29 with the electronics circuits necessary according to the present invention. It will further be apparent that the loudspeaker 28 is secured on that side of the circuit card 29 which is turned: to face in towards the interior of the protector hood 2. Around the loudspeaker 28, there is disposed a sealing or gasket ring 30 which is intended to seal against the outside of a partition in the hood, where this partition corresponds to the bottom wall 25 in the recess 23 illustrated in FIG. 4. The loudspeaker has a number of apertures 31 through which the sound produced by the loudspeaker may pass to the interior of the protector hood 2. On the rear side of the membrane of the loudspeaker, there is a minor volume which to some degree damps the base range of the sound emitted by the loudspeaker.

It will be apparent from FIG. 6 that the wall 25 which forms the bottom of the recess 23 which is intended for accommodating the unit 6 from the outside of the hood is closed towards the inside of the hood 2. It will be particularly apparent that the opening 32 through which the loudspeaker 28 extends displays a close fit with the periphery of the loudspeaker. Since the sealing ring 30 on the circuit card 29 connects around the loudspeaker 28 to the outside of the wall 25, it will be readily perceived that the sealing-off of the interior of the hood to the ambient surroundings will be thorough.

It will further be apparent from FIG. 6 that the loudspeaker 28 extends in a considerable distance into the interior of the hood 2 so that its sound apertures 3 1 will be located proximal the opening of the acoustic meatus of the wearer of the hearing protector.

In one embodiment, where the microphone 7 is turned to face forwards, it is suitably designed with superior directional effect so that the pick-up lobe of the microphone may be directed towards the face of a person standing in front of and speaking to the wearer of the hearing protector. As a result, it is possible to restrict the take-up of ambient noise via the microphone 7 and thereby increase the level of understanding in the communication between the two people.

The hearing protector of the invention has thus far been described with reference to the earmuff assembly embodiment of FIGS. 1-6. This embodiment is, of course, merely exemplary and in no way limits the scope of the invention. The hearing protector of the invention may embody any form or configuration suitable for providing the passive damping and selective communication features discuss herein.

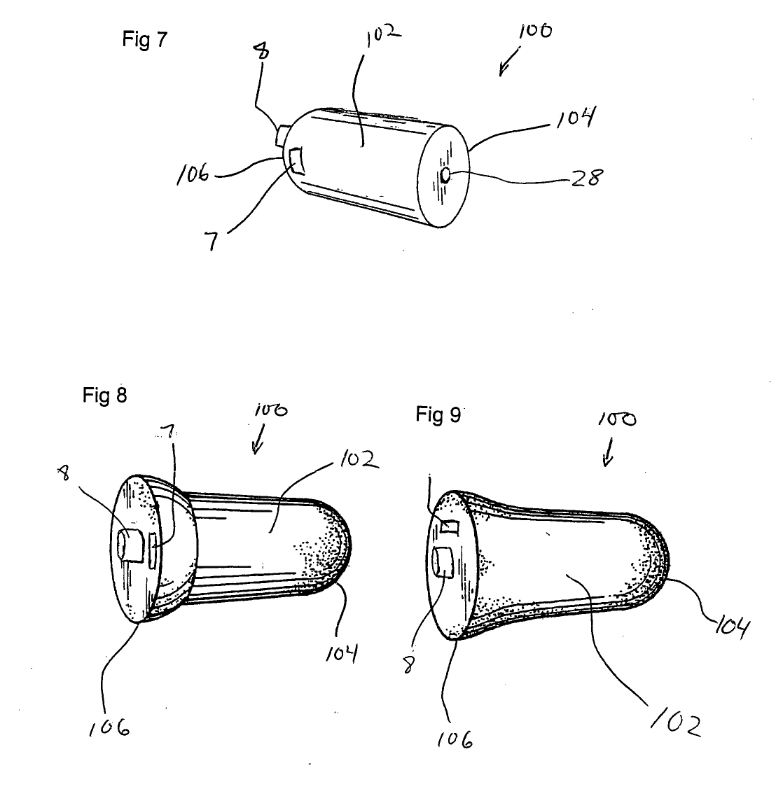

FIGS. 7-12 include hearing protector devices in additional embodiments of the invention. Identical parts are represented herein and throughout the drawings with consistent reference numerals.

FIG. 7 shows a hearing protector device 100 including a generally cylindrical body 102 having a front end 104 and an opposing rear end 106. The loudspeaker 28 is disposed on or at least partially within the body 102 proximate to the front end 104. The loudspeaker 28 may be exposed at the front end 104 or may alternatively be submerged within the body 102. Here, the loudspeaker 28 is shown to be of a smaller size than that shown in earlier drawings. However, the loudspeaker 28 performs and operates as discussed above. The microphone 7 is disposed on or at least partially within the body 102 proximate to the rear end 106. The microphone is shown here and in the remaining drawings in schematic form to generally represent any suitable sound receiving mechanism as discussed herein in the context of the present invention. The activator button 8 described above is disposed at the rear end 106 of the body 102 so as to be easily accessible by the user. The body 102 is composed of any compressibly, resilient material such as, for example, a foam, a rubber, a plastic, etc.

In use, the front ends of hearing protectors 100 are inserted at least partially into the respective ear canals of a user. The body 102 occludes the earcanal and attenuates the passage of sound. As similarly discussed above, when the user manipulates the activation button 8, ambient sound is received by the microphone 7, amplified by the amplifier (not shown), and projected by the loudspeaker 28 to the earcanal of the user. In a preferred embodiment, frequencies corresponding to the range of human speech (e.g., 125-6000 Hz) are amplified by the amplifier and projected by the loudspeaker 28. Also, as discussed above, this amplification varies so as to maintain the total level of sound (ambient noise passing through the attenuating body 102 and sound selectively projected to the ear by the microphone, amplifier, loudspeaker) below a predetermined maximum sound level. For example, if the ambient noise is very loud, the frequencies are projected by the loudspeaker at a reduced level to keep the total sound beneath the predetermined maximum. The electronic unit of the protector 100 may be deactivated as discussed above by the user releasing the activation button 8, or automatically after a predetermined time period, etc.

FIGS. 8 and 9 show the hearing protector 100 where the body 102 includes alternative shapes. These embodiments of the protector 100 function and operate as discussed with reference to FIG. 7. In FIGS. 8 and 9, the microphone 7 is shown as disposed on the rear end 106.

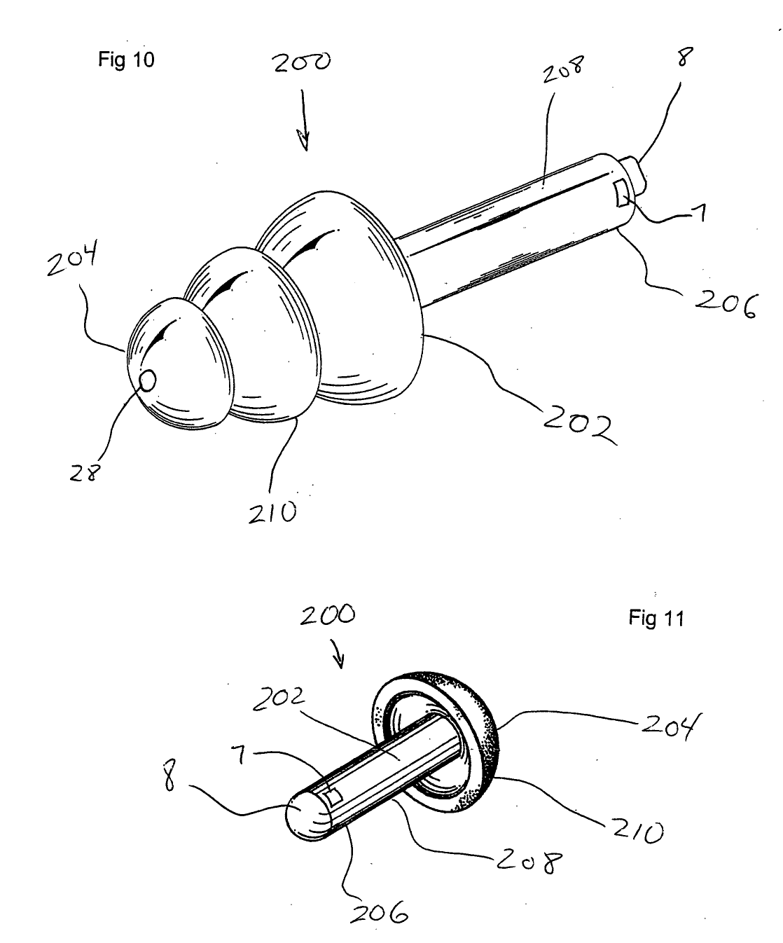

FIGS. 10 and 11 show a hearing protector 200 in another embodiment of the invention. Similar to the hearing protector 100 of FIGS. 7-9, the protector 200 includes a body 202 having opposing front and rear ends 204 and 206 with the loudspeaker 28 disposed at the front end 204 and the activation button 8 and microphone 7 disposed at the rear end 206. The body 202 includes a stem component 208 and at least one hemispherical or semi-hemispherical flange element 210 disposed thereon.

Here again, in use, the front end 204 of the body 202 is inserted at least partially into the ear canal of a user. The at least one flange 210 occludes the earcanal and attenuates the passage of sound. The user may selectively manipulate the button 8 to activate the electronic unit of the protector 200 such that sound is received by the microphone, a certain frequency range of which is then variably amplified by the amplifier and projected by the loudspeaker so as to be heard by the user. Again, the protector 200 provides that the total sound level experienced by the user is less than a predetermined maximum.

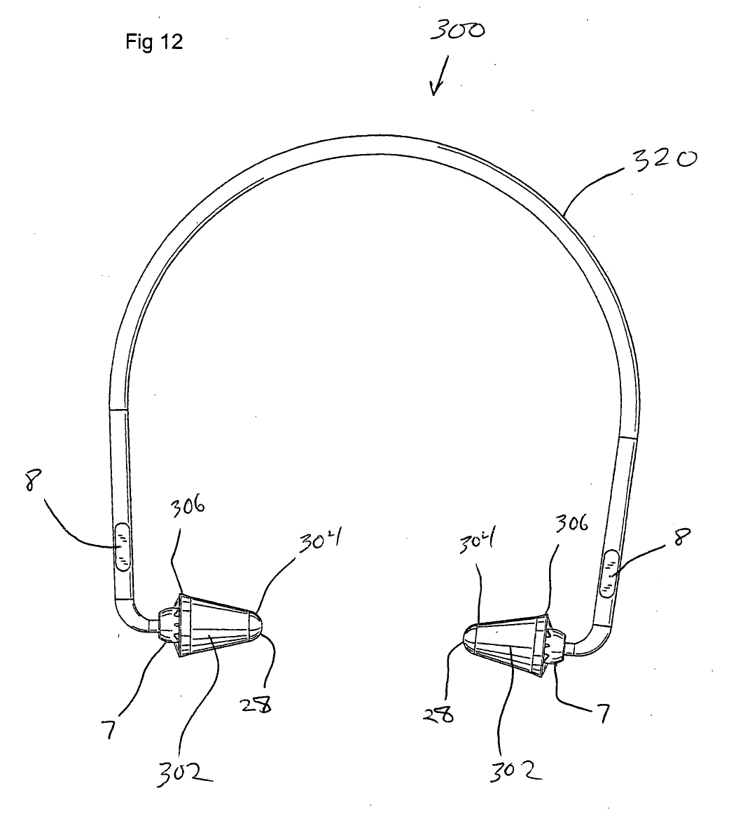

FIG. 12 shows a hearing protector 300 in an additional embodiment of the invention. The protector 300 includes two attenuating bodies 302 affixed to a band 320 intended to rest on the head or neck or beneath the chin of a user. The bodies 302 include opposing front and rear ends 304 and 306 and include the microphone 7, activation button 8, and loudspeaker 28 generally as discussed with regard to the previous embodiments. Here, the body 302 is shown as generally having a conical-like shape. The microphones 7 are represented as rounded features at the rear ends 306 of the bodies 302. A button 8 is disposed on the band 320 proximate to each of the bodies 302. More generally, the protector 300 may include one or more activation buttons disposed on the band 320 and/or at least one of the attenuating bodies 302.

The hearing protector 300 functions and operates similar to the protectors of FIGS. 7-11. That is, the front ends 304 are received at least partially within respective ear canals of the user where they attenuate the passage of sound. The electronic unit of the protector 300 may be activated by the button 8 to allow selected frequencies to be heard where the total sound level experienced by the user is kept beneath a predetermined maximum.

Any of the protectors 100 and 200 may be used in conjunction with the band 300, as desired. More generally, any two hearing protector bodies 102, 202, 302 may be connected by a band, cord, cable, etc.

Herein, a single microphone 7 and a single loudspeaker 28 as been discussed with regard to each of the attenuating bodies 102, 202, and 302. However, each body may include multiple microphones 7 and/or multiple loudspeakers 28, as desired. Additionally, the microphones 7 and/or loudspeakers 28 may be disposed within the bodies 102, 202, 302 and/or on an exterior surface thereof. In another embodiment, the microphone may be disposed remotely from the body 102, 202, 302. For example, the microphone may be affixed to clothing of the user, such as a jacket, helmet, safety glasses, etc., and may wirelessly send sound signals to the amplifier and other electronic circuitry of the body 102, 202, 302, or of the unit 6 discussed above with reference to the protector 1. Alternatively, the microphone may be disposed on a wire which is tethered to the body 102, 202, 302 or to the unit 6. The invention includes any such desirable configuration of the microphone and loudspeaker. Similar variations of the amplifier and other circuitry of the disclosed hearing protectors are within the broad scope of the invention.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A hearing protector comprising:
a hearing hood with passive noise damping;
a microphone disposed exteriorly on the hearing protector;
a loudspeaker disposed inside the hood; and
an amplifier for amplifying sound signals caught by the microphone and passing the signals on to the loudspeaker;
wherein
the noise damping of the hearing hood is broad-band,
the amplifier has variable amplification and frequency range which corresponds to the frequency range of human speech, and
the amplifier has a maximum predetermined amplification where the sum total of the sound levels that are caused, on the one hand by ambient sound passing through the hood, and on the other hand by sound emitted by the loudspeaker, amounts to a maximum predetermined value.

2. The hearing protector as claimed in claim 1, wherein the amplifier is manually engageable.

3. The hearing protector as claimed in claim 1, further comprising a timer for disengaging the amplifier after a predetermined period of time.

4. The hearing protector as claimed in claim 3, wherein the amplifier is manually disengageable.

5. The hearing protector as claimed in claim 1, wherein the amplifier, the microphone, the loudspeaker, and a battery are disposed together as a replaceable unit.

6. The hearing protector as claimed in claim 5, wherein the unit is removably disposed in a recess in the hearing hood, a partition between the recess and the interior of the hearing hood having an aperture for the loudspeaker.

7. The hearing protector as claimed in claim 1 to 6, wherein the loudspeaker extends into an interior of the hearing hood so as to be close to an ear of a wearer of the hearing protector.

8. A hearing protector device, comprising:
a sound attenuating body; and
an electronic unit having a microphone, an amplifier, and a loudspeaker;
wherein the electronic unit is selectively activated and, when activated, receives sound from an ambient environment, amplifies a frequency range of the received sound corresponding to a frequency range of human speech, and varies said amplification such that a total level of sound passing through the body and through the electronic unit does not exceed a maximum predetermined sound level.

9. The hearing protection device of claim 8, wherein the sound attenuating body comprises a two earmuff cups placeable over the ears of a user, wherein the microphone is a multi-directional microphone disposed on an exterior of at least one of the earmuff cups, wherein the loudspeaker is disposed within an interior of at least one of the earmuff cups, said earmuff cups connected by a head band.

10. The hearing protection device of claim 8, wherein the sound attenuating body comprises an compressibly resilient earplug.

11. The hearing protection device of claim 10, wherein the earplug comprises a cylindrical foam earplug.

12. The hearing protection device of claim 11, wherein the loudspeaker and microphone are disposed at least partially within the foam earplug.

13. The hearing protection device of claim 11 wherein the loudspeaker is disposed at least partially within the earplug and the microphone is disposed remotely from the earplug.

14. The hearing protection device of claim 8, wherein the earplug includes a stem portion and a plurality of hemispherical or semi-hemispherical flange elements disposed on the stem portion.

15. The hearing protection device of claim 8, wherein the sound attenuating body comprises two earplugs connected by a band.

16. The hearing protection device of claim 8, wherein the microphone is disposed on the band.

17. The hearing protection device of claim 8, further comprising an activation button for manually activated the electronic unit.

18. The hearing protection device of claim 17, wherein the body comprises an earplug, the electronic unit is disposed within the earplug, and the activation button extends from the earplug.
